(19) United States
(12) Patent Application Publication
(10) Pub. No.: US 2002/0187960 A1
(43) Pub. Date: Dec. 12, 2002

(54) Method for inhibiting microbial growth in aqueous cyclodextrin compositions and the resulting composition

(76) Inventors: Chris Sikorski, Schererville, IN (US); Roland Beck, Valparaiso, IN (US)

Correspondence Address: BIERMAN MUSERLIAN AND LUCAS, 600 THIRD AVENUE, NEW YORK, NY, 10016

(21) Appl. No.: 09/843,183

(22) Filed: Apr. 26, 2001

(51) Int. Cl. A61K 33/00; A61K 31/724

(52) U.S. Cl. 514/58; 424/722

(57) ABSTRACT

Microbial growth in an aqueous cyclodextrin composition is inhibited by the addition of a small, polar base to the composition so as to provide a pH of 10-14 to the composition. The base includes sodium potassium, calcium, magnesium and ammonium hydroxides and carbonates as well as ethylamine, diethylamine and triethylamine. The composition is the resulting alkalaine, aqueous cyclodextrin solution. Adjusting the pH to the alkaline side shows superior antimicrobial action compared to conventional antimicrobial agents.

### BACKGROUND OF INVENTION

1. Technical Field

The present invention relates to a method for inhibiting microbial growth in aqueous compositions which contain cyclodextrins and especially aqueous cyclodextrin compositions which are intended for use in food, pharmaceutical and cosmetic compositions.

2. Description of Related Art

Cyclodextrins, also called Schardinger dextrins, cycloamyloses, cyclomaltoses and cycloglucans, are polymers of anhydroglucose units in the form of a ringed structure. A ring of six anhydroglucose units is called alpha cyclodextrin while seven and eight membered rings are referred to as beta and gamma cyclodextrin, respectively.

Cyclodextrins are produced by treating starch or liquefied starch with an enzyme, cyclodextrin glycosyl-transferase (CGT), at the appropriate pH, temperature, and time for the selected CGT. The starch may be from any selected plant variety. The enzyme CGT is obtained from microorganisms such as B. macerans, B. megaterium, B. circulars, B. stearothermophilus and Bacillus sp. (alkaliophilic), as well as others. The parameters for the reaction between the selected CGT and the selected starch are conventional and well described in the literature. Cyclodextrins can also be further modified by further reaction with enzymes or chemicals.

Cyclodextrins have a wide variety of uses in pharmaceuticals, agricultural chemicals, cosmetics, and foods. These uses occur because of the natural ability of the cyclodextrin to act as a clathrate and encapsulate other chemical compounds. The encapsulated compound is referred to as a guest molecule, and the cyclodextrin as the host. Cyclodextrin has a hydrophilic exterior which makes it soluble in water and a hydrophobic interior which accepts a water insoluble guest and allows the cyclodextrin to solubilize the guest in water.

Typically, the cyclodextrin is stored and/or transported in an aqueous solution. Often, the aqueous cyclodextrin solution is then employed in encapsulating the guest and formulating a food, cosmetic or pharmaceutical composition. A problem associated with aqueous compositions which contain cyclodextrin is that microbes can grow therein, thereby contaminating the composition. Cyclodextrin, because it is a polymer of glucose, can act as a food source for the microbe. This microbial growth is especially problematic in the aqueous cyclodextrin composition which is in storage waiting for encapsulation and formulation and during transportation of the aqueous cyclodextriun composition between the cyclodextrin producer and the producer of the food, cosmetic or pharmaceutical composition. It is not untypical for the aqueous cyclodextrin composition to be stored for a period of one or two weeks.

Attempts to inhibit or prevent the growth of microbes in aqueous cyclodextrin compositions have focused on complex antimicrobial agents. Examples of such antimicrobial agents include a combination of boric acid and halide compound or a specific quaternary ammonium compound, see U.S. Pat. No. 5,985,310; hydrophobic antimicrobial agents, see U.S. Pat. No. 5,861,145; organic sulfur compounds, 3-Isothiazolone compounds, sodium pyrithione, halogenated compounds, cyclic organic nitrogen compounds, aldehydes, quaternary compounds, dehydroacetic acid, and phenyl compounds, see U.S. Pat. No. 5,534,165; phenol derivatives, see EPO 119737; chlorohexidine derivatives, see JP 60149530; cationic surfactant, see JP 01016728; and diclofenac sodium, a cationic surfactant, paraben or an alcohol, see JP 6016547.

A number of problems are associated with the use of such antimicrobial agents with aqueous cyclodextrin compositions. Antimicrobial agents can complex with the cyclodextrin thereby causing a loss in the antimicrobial activity of the antimicrobial agent. This complexation reaction between the antimicrobial agent and the cyclodextrin also interferes with the cyclodextrins ability to complex. Furthermore, there has been concern that the widespread use of antimicrobial agents causes the development and proliferation of microbes which are resistant to conventional antimicrobial agents. Additionally, in certain instances, the antimicrobial agent must be deactivated with another chemical agent thereby yielding potentially undesirable odors and by-products. Furthermore, certain of the antimicrobial agents are toxic to humans, animals and plants at low levels.

There is a need for a simple method for inhibiting microbial growth in aqueous cyclodextrin compositions which avoids the problems associated with antimicrobial agents normally employed with such compositions.

### SUMMARY OF INVENTION

It has now been discovered that if a small, polar base is used in an aqueous cyclodextrin composition in an amount sufficient to cause the composition to have an alkaline pH, that microbial growth in the composition is inhibited.

The use of a small, polar base provides a number of advantages over conventional antimicrobial agents. First, small, polar bases do not readily complex with the cyclodextrin. Thus, the potency of the base is unaffected by the cyclodextrin. Second, although an alkaline pH provides a hostile environment to a broad spectrum of microbes, it is generally non-toxic, at low levels, to humans and other animals and plant life. Third, when necessary, the base is easily neutralized with an acid resulting in the formation of a simple salt. Fourth, small, polar bases are generally inexpensive and readily available compared to conventional antimicrobial agents. Fifth, an alkaline pH aids in solubilizing and in reducing the viscosity of cyclodextrin in water thereby increasing the efficiency of both handling and transportation of the composition.

Broadly, the present invention is a method for inhibiting the growth of microbes in aqueous cyclodextrin compositions which comprise providing an effective amount of a small, polar base to the composition so as to insure an alkaline pH to the composition.

Broadly, the composition of the present invention is an aqueous cyclodextrin composition which contains a small, polar base in an amount effective to insure that the composition has an alkaline pH and to inhibit microbial growth.

The resulting composition, an alkaline, aqueous cyclodextrin solution, can be used directly in a final product where the pH of the solution is not detrimental to the final product or its use. There may, however, be a need to neutralize the composition. In the case of a food, cosmetic or pharmaceutical product, it is preferred that the alkaline, aqueous cyclodextrin solution be subject to neutralization by adding an acid to the solution in an amount effective to adjust the pH to neutral. The acid should be one that is Generally Recognized as Safe (GRAS) where the final product is a food, cosmetic or pharmaceutical product.

### DETAILED DESCRIPTION OF INVENTION

Suitable bases for use in the present invention are small, polar inorganic and organic bases. Such inorganic bases include hydroxides and carbonates of alkaline metals and alkaline earth metals. More particularly, sodium, potassium, calcium and magnesium hydroxides and carbonates. Suitable inorganic bases include sodium hydroxide (NaOH), potassium hydroxide (KOH), calcium hydroxide (Ca(OH)₂), magnesium hydroxide (Mg(OH)₂), sodium carbonate (Na₂CO₃), potassium carbonate (K₂CO₃) calcium carbonate (Ca(CO₃)₂), magnesium carbonate (MgCO₃), ammonium carbonate (NH₄)₂CO₃), and ammonium hydroxide (NH₄OH). Sodium hydroxide is preferred.

Suitable organic bases include triethylamine ((C₂H₅)₃N), diethylamine (C₂H₅)₂NH) and ethylamine (C₂H₅NH₂)

A single base or a plurality of bases can be used to provide the alkaline pH of the composition.

The term “small” is used to describe the physical structure of the base because the molecular structure of the base is small in physical size compared to the physical size of the cyclodextrin and to other more complex and larger bases.

The term “polar” is used to describe the base because of the ability of the base to disassociate in solution and form a charge.

The amount of base added to the composition is sufficient to cause the composition to have an alkaline pH. Thus, the amount of base added is an effective amount, effective to insure that the composition has an alkaline pH. More specifically, the pH of the composition should be equal to or greater than about 10 and, more preferably, equal to or greater than about 11 with good results being obtained at a pH of about 12. Suitably, the pH of the composition is about 10 to about 14 and, more preferably, about 11 to about 13.

The pH of the solution is determined in a conventional manner using conventional equipment, for example, a pH meter.

The concentration of the base in the composition is suitably low, less than about 5% by weight based on the overall weight of the composition.

The actual amount of base used in the composition depends on the pH of the composition before the addition of the base. The less alkaline the composition, the more base that must be used to insure that the composition has an alkaline pH. The more alkaline the composition is before the addition of the base, the less base that must be added to the composition to insure that the composition has an alkaline pH.

Providing the base to the solution is suitably done by adding the base to the solution. Adding the base to the aqueous composition can be accomplished in any conventional manner using conventional equipment. For example, during the initial mixing of the composition, the base is added in an amount effective to provide an alkaline pH and inhibit or prevent microbial growth.

The term “cyclodextrin” as used herein means both uncomplexed and/or complexed cyclodextrins as well as modified, unmodified, polymerized and branched cyclodextrins. Alpha-, beta- or gamma-cyclodextrins as well as modified or derivatives of these cyclodextrins are conventionally used in aqueous compositions. Hydroxypropylated cyclodextrins and methylated cyclodextrins are common modified cyclodextrins which are used in commercial aqueous compositions which contain cyclodextrins.

The aqueous cyclodextrin composition can contain a simple cyclodextrin or mixture of different types of cyclodextrins.

Microbes include both fungi and bacteria.

If necessary, the pH of the composition can be tested after storage of the composition for a period of time and additional base added so as to maintain the pH of the system on the alkaline side in accordance with the present invention. Compositions made in accordance with the present invention, however, have been found to be stable over periods of time (e.g. 5 days) and not in need of further additions of base.

Furthermore, both temperature and pressure of the composition do not markedly effect the ability of the base to fight microbe growth. Thus, the present invention provides a stable method for preventing or inhibiting growth of microbes in aqueous cyclodextrin compositions over long periods of time.

The alkaline, aqueous cyclodextrin composition can be used directly in formulating a final product or it can be neutralized. There is no need to neutralize the composition if it is used in a final product that has an alkaline pH. The alkaline composition of the present invention is suitable for use directly in formulations of cleaners, paints, coating removal compositions, anti-microbial compositions, antifungal compositions, polishing compositions, deodorizing compositions, as well as other alkaline compositions.

If the alkaline composition needs to be neutralized, any acid can be employed provided it does not have a deleterious effect on the composition or the final product. Suitable acids include hydrochloric, citric, phosphoric, tartaric, sulfuric, and acetic. Preferably, the pH is adjusted to about 6 to about 7 by the acid addition.

The acid is added and mixed with the alkalaine solution in a conventional manner using conventional equipment so as to obtain a neutralized solution.

These and other aspects of the present invention may be more fully understood by reference to one or more of the following examples.

### EXAMPLE 1

This example illustrates preparing an aqueous hydroxypropylated beta-cyclodextrin composition in accordance with the present invention.

To make up the aqueous cyclodextrin solution having an alkaline pH, 50 grams of hydroxypropylated beta-cyclodextrin (dry basis), 1.5 grams of sodium hydroxide (dry basis), and a sufficient amount of water to provide a total of 100 grams of solution were mixed together. The resulting solution had a pH of greater than 11.6.

In this example, the hydroxypropylated beta-cyclodextrin was in powder form prior to mixing it with the other components.

### EXAMPLE 2

This example illustrates another method for preparing an aqueous hydroxypropylated beta-cyclodextrin composition in accordance with the present invention.

To make up the aqueous composition, 56 kg of water and a minimum of about 2 Kg of 50% sodium hydroxide are added to a 50-gallon reactor with an impeller. Beta-cyclodextrin (100 kg as-is, 88 kg dry basis) is added gradually to the stirring alkaline solution. The reactor is sealed, purged with nitrogen, and heated to reaction temperature. An appropriate amount of propylene oxide is then piped into the reactor gradually in order to control the reactor pressure. Upon completion of the propylene oxide addition, the reactor is maintained at reaction temperature and then stirred until the reaction is completed. The resulting clear cyclodextrin solution has a ph greater than 11.

In this example, the hydroxypropylated beta-cyclodextrin is prepared from unmodified cyclodextrin and the resulting aqueous composition has an alkaline pH in accordance with the present invention.

### EXAMPLE 3

This example illustrates the superior results obtained with the present invention compared to a conventional antimicrobial agent.

Aqueous solutions of hydroxypropylated beta-cyclodextrin were prepared, each of which contained approximately 40% by weight hydroxypropylated beta-cyclodextrin, approximately 60% by weight water and a remainder of either base or antimicrobial agent. The amount of base employed was effective to raise the pH to approximately 12 as shown in the Table below while the amount of antimicrobial agent added was 10 ppm as reported in the Table below.

Total Plate Count g/CFU

| | | | | Antimicrobial |
|---|---|---|---|---|
| Sample | 48 hrs. | 120 hrs. | pH | Agent |
| A | <20 | <20 | 12.4 | 0 |
| B | <20 | <20 | 12.4 | 0 |
| C | <20 | <20 | 12.2 | 0 |
| D | <20 | 60 | 7.5 | 10 ppm |
| E | <20 | 60 | 7.4 | 10 ppm |
| F | <20 | 500 | 7.5 | 10 ppm |

The plate count was conducted in a conventional manner as listed in the Corn Refining Association method book. The pH was measured using a pH meter and did not vary during the period of testing.

As can be seen, the present invention inhibited microbial growth for 5 days while the antimicrobial agent allowed growth to occur.

The antimicrobial agent employed in this example was Kathon.CG®, a mixture of about 77% 5-chloro-2-methyl-4-isothiazolin-3-one and about 23% 2-methyl-4-isothiazolin-3-one, sold by Rohm and Haas Company.

It will be understood that the claims are intended to cover all changes and modifications of the preferred embodiments of the invention herein chosen for the purpose of illustration which do not constitute a departure from the spirit and scope of the invention.

What is claimed is:

1. A method for inhibiting microbial growth in an aqueous cyclodextrin composition comprising providing an effective amount of a small, polar base to said composition so as to insure said composition has an alkaline pH.

2. The method of claim 1 wherein said alkaline pH is equal to or greater than about 10.

3. The method of claim 1 wherein said alkaline pH is equal to or greater than about 11.

4. The method of claim 1 wherein said alkaline pH is about 10 to about 14.

5. The method of claim 1 wherein said alkaline pH is about 11 to about 13.

6. The method of claim 1 wherein said base is an hydroxide or a carbonate of an alkaline metal or alkaline earth metal.

7. The method of claim 1 wherein said base is selected from the group consisting of sodium hydroxide, potassium hydroxide, calcium hydroxide, magnesium hydroxide, ammonium hydroxide, sodium carbonate, potassium carbonate, calcium carbonate, magnesium carbonate, ammonium carbonate, ethylamine, diethylamine and triethylamine.

8. The method of claim 1 wherein said composition is stored, and then neutralized for use in a food, pharmaceutical or cosmetic composition.

9. The method of claim 1 wherein said cyclodextrin is a modified or unmodified cyclodextrin.

10. In a composition comprising water and cyclodextrin, the improvement providing adding an effective amount of a small, polar base to said composition so as to insure said composition has an alkaline pH and microbial growth is inhibited.

11. The method of claim 10 wherein said alkaline pH is equal to or greater than about 10.

12. The method of claim 10 wherein said alkaline pH is equal to or greater than about 11.

13. The method of claim 10 wherein said alkaline pH is about 10 to about 14.

14. The method of claim 10 wherein said alkaline pH is about 11 to about 13.

15. The method of claim 10 wherein said base is an hydroxide or a carbonate of an alkaline metal or an alkaline earth metal.

16. The method of claim 10 wherein said base is selected from the group consisting of sodium hydroxide, potassium hydroxide, calcium hydroxide, magnesium hydroxide, ammonium hydroxide, sodium carbonate, potassium carbonate, calcium carbonate, magnesium carbonate, ammonium carbonate, ethylamine, diethylamine and triethylamine.

17. The method of claim 10 wherein said composition is stored, and then neutralized for use in a food, pharmaceutical or cometic composition.

18. The method of claim 10 wherein said cyclodextrin is a modified or unmodified cyclodextrin.

19. A composition of cyclodextrin comprising water, cyclodextrin and a preservative for inhibiting microbial growth, the improvement comprising said preservative consisting essentially of a base in an amount effective to adjust the composition to an alkaline pH.

20. The composition of claim 19 wherein said pH is about 10 to about 14.
